(19) United States
(12) Patent Application Publication
(10) Pub. No.: US 2025/0000799 A1
(43) Pub. Date: Jan. 2, 2025

(54) Reconstitutable, Single Use Antidiabetic Compositions

(71) Applicant: V-Ensure Pharma Technologies Private Limited, Navi Mumbai (IN)

(72) Inventors: Sathyanarayana Vemula, Navi Mumbai (IN); Saswat Abhishek Padhi, Navi Mumbai (IN); Gurbasish Pradhan, Navi Mumbai (IN)

(21) Appl. No.: 18/708,360

(22) PCT Filed: Feb. 4, 2022
(86) PCT No.: PCT/IB2022/050971

§ 371 Date: May 8, 2024

(30) Foreign Application Priority Data
Nov. 11, 2021 (IN) 202121051628

(51) Int. Cl. A61K 9/16; A61K 9/00; A61K 31/155

(52) U.S. Cl. CPC A61K 9/1652; A61K 9/0095; A61K 9/1611; A61K 9/1623; A61K 9/1635; A61K 9/1676; A61K 31/155

(57) ABSTRACT

The present invention relates to reconstitutable, single use antidiabetic compositions which provide a single use composition comprising a) metformin or pharmaceutically acceptable salt as an extended release composition, b) optionally one or more antidiabetic agent(s) and optionally pharmaceutical acceptable excipients; wherein the single use composition is reconstituted just before consumption. It also covers the process of preparation of said reconstitutable, single use antidiabetic compositions.

### PRIORITY

This application claims the benefit of Indian complete application No. 202121051628 dated 11 Nov. 2021 entitled, ‘Reconstitutable, single use antidiabetic compositions’, the contents of which are incorporated herein by reference.

### TECHNICAL FIELD OF THE INVENTION

The present invention relates to reconstitutable, single use antidiabetic compositions which provide a single use composition comprising a) metformin or pharmaceutically acceptable salt as an extended release composition, b) optionally one or more antidiabetic agent(s) and optionally pharmaceutical acceptable excipients: wherein the single use composition is reconstituted just before consumption. The present invention also relates to dose uniformity of antidiabetic agent(s) is as per USP, wherein dose accuracy of antidiabetic agent(s) per dose is between 95% and 105%. It also covers the process of preparation of said reconstitutable, single use antidiabetic compositions.

### BACKGROUND OF THE INVENTION

Metformin is an insulin sensitizer which is chemically dimethyl biguanide compound. It is the most widely used medication for diabetes. Metformin is an antihyperglycemic agent which improves glucose tolerance in patients with type 2 diabetes mellitus, lowering both basal and postprandial plasma glucose. Metformin decreases hepatic glucose production, decreases intestinal absorption of glucose, and improves insulin sensitivity by increasing peripheral glucose uptake and utilization. The Chemical Structure of Metformin hydrochloride is as follows:

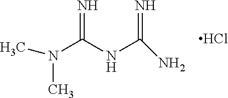

The chemical name of metformin hydrochloride is N, N-dimethylimidodicarbon-imidic diamide hydrochloride. Metformin hydrochloride. USP is a white crystalline powder with a molecular formula of C₄H₁₁N₅·HCl and a molecular weight of 165.62. It is freely soluble in water, slightly soluble in alcohol; practically insoluble in acetone and in methylene chloride.

Metformin is marketed in currently marketed under various dosage forms such as immediate release tablets, an extended release tablets. Glucophage® is an immediate release tablet contains 500 mg, 850 mg and 1000 mg having tablet size: 11 mm, 13 mm, 19 mm respectively and Glucophage® needs to administer twice daily. Similarly, Glucophage XR® which is an extended release tablet contains 500 mg and 750 mg both are having tablet size 19 mm and needs to administer once a daily. Glucophage® and Glucophage XR® tablets are larger in size, hence patients having difficulty in swallowing the large size tablets wherein Glucophage XR® is described in U.S. Pat. No. 6,475,521, which relates to a method for preparing a biphasic controlled release delivery system adapted for delivery of metformin.

Fortamet® is also an extended release tablet contains for 500 mg and 1000 mg having tablet size 12 mm & 13 mm respectively. Fortamet® listed patents U.S. Pat. No. 6,866,866 discloses controlled release oral dosage form for the reduction of serum glucose levels in human patients with NIDDM, comprising an effective dose of metformin or a pharmaceutically acceptable salt thereof and U.S. Pat. No. 6,790,459 method for lowering blood glucose levels in human patients needing treatment for non-insulin-dependent diabetes mellitus (NIDDM) using metformin.

Glumetza® is an extended release tablet having 500 mg and 1000 mg having large tablet size 18 mm and 20 mm respectively. For Glumetza® 500 mg listed patent is U.S. Pat. No. 6,723,340 discloses metformin hydrochloride controlled-release tablet solid monolithic matrix comprising a combination of poly(ethylene oxide) and hydroxypropyl methylcellulose. For Glumetza® 1000 mg listed patents are U.S. Pat. No. 7,780,987 & U.S. Pat. No. 8,323,692 discloses stable controlled release monolithic coating compositions of metformin hydrochloride for use in coating pharmaceutical oral dosage forms comprising a polyglycol having a melting point greater than 55° C. and an aqueous dispersion of a neutral ester copolymer lacking functional groups. Therefore, metformin marketed under the trade names such as Glucophage® immediate release tablet, Glucophage XR®, Glumetza® & Fortamet® extended release tablets are larger in size, hence patients having difficulty in swallowing the large size tablets. The problem of patient compliance still remains.

Further, RIOMET® is an immediate release oral solution contains 500 mg/5 ml. For RIOMET® listed U.S. Pat. No. 6,890,957 discloses liquid pharmaceutical composition comprises metformin, sweetener, polyhydroxy alcohol, a mineral acid and bicarbonate salt. RIOMET® pack contains metformin hydrochloride oral solution bottle with specific dosing cup. Due to multiple dosing administration of RIOMET® patient needs to carry bottle with specific dosing cup, which is inconvenient to the patient.

RIOMET ER® is an extended release oral suspension contains 500 mg/5 ml. For RIOMET ER® listed U.S. Pat. No. 9,962,336 discloses method for preparing a stable extended release suspension composition comprising multiple coated cores of an active ingredient by using a suspension base, wherein the suspension base ensures substantially similar in-vitro dissolution release profile of the active ingredient upon storage of the suspension compositions for at least seven days. RIOMET ER® pack contains drug pellets bottle and drug diluent bottle along with dosing cup. For administration of RIOMET ER® patient needs to carry both bottles with dosing cup, which is inconvenient to the patient. Furthermore, due to stability reasons patient needs to dispose of any unused portion of the reconstituted suspension of RIOMET ER® in the household trash after 100 days.

Hence, there is a need to make reconstitutable, single use antidiabetic composition, for better patient compliance, to avoid multiple daily dosing, ease of dosing administration and those who cannot swallow the solid dosage form.

The present invention relates to reconstitutable, single use antidiabetic compositions which include metformin or pharmaceutically acceptable salt as an extended release composition, optionally one or more antidiabetic agent(s) and optionally pharmaceutical acceptable excipients; wherein the single use composition is reconstituted just before consumption, consequently the present invention eradicate the problem of patient compliance. Further, the reconstitutable, single use antidiabetic composition remains stable over the long period of time.

### SUMMARY OF THE INVENTION

The present invention relates to reconstitutable, single use antidiabetic compositions which provide a single use composition comprising a) metformin or pharmaceutically acceptable salt as an extended release composition, b) optionally one or more antidiabetic agent(s) and optionally pharmaceutical acceptable excipients; wherein the single use composition is reconstituted just before consumption.

Another aspect of the invention, the composition is devoid of osmogent. More precisely, the present invention particularly relates to single use composition comprising
a. metformin or pharmaceutically acceptable salt as an extended release composition as portion 1
b. external phase as portion 2 optionally one or more antidiabetic agent(s):
wherein extended release portion 1 and portion 2 are filled into the single dose sachet at different stages.

Further aspect of the present invention is to provide a single use pharmaceutical composition comprising
a. metformin or pharmaceutically acceptable salt as an extended release composition
b. optionally one or more antidiabetic agent(s); and

wherein dose uniformity of active ingredients is as per USP.

In another aspect of the present invention, there is provided a single use composition comprising
a. metformin or pharmaceutically acceptable salt as an extended release composition
b. optionally one or more antidiabetic agent(s); and

wherein dosing accuracy for active ingredients per dose is between 95% and 105%.

The present invention also relates to a process for preparation of said reconstitutable, single use antidiabetic compositions.

### DETAILED DESCRIPTION OF THE INVENTION

The detailed description of the present invention described hereinafter.

The present invention relates to reconstitutable, single use antidiabetic compositions which provide a single use composition comprising a) metformin or pharmaceutically acceptable salt as an extended release composition, b) optionally one or more antidiabetic agent(s) and optionally pharmaceutical acceptable excipients; wherein the single use composition is reconstituted just before consumption.

The term “reconstitution” used herein means the process of adding a diluent to a dry ingredient to make it a liquid.

The term “composition” used herein means that it is a pharmaceutical formulation which is suitable for administration to a patient. Other terms such as “formulation” or “dosage form” are used herein interchangeably.

The terms “extended release” used herein means the active agent is released at a predetermined rate that is different or slower than immediate release. Other terms such as “controlled release” or “sustained release” or “modified release” or “prolonged release” have been used interchangeably.

The term “single use composition” means the composition to be consumed in single dose. The examples of single use compositions includes, but are not limited to tablets, capsules, pellets, sachets, powders, granules, and lozenges.

The abbreviation “USP” used herein means United States Pharmacopeia.

In one aspect of the present invention, reconstitutable, single use antidiabetic composition is in the form of pellets, beads, granules, powders, spheroids or tablets and mixture thereof.

In another embodiment of the present invention, the conventional, extended release or seal coating of the said dosage form done by using polymers selected from the group consisting of cellulosic polymers such as ethyl cellulose, hydroxypropylmethylcellulose; Surelease ARC; hydroxypropylcellulose, cellulose acetate, polyvinyl alcohol and combination thereof.

In another aspect of the invention reconstitutable, single use antidiabetic composition may be in the form of tablets such as dispersible tablets, film coated tablets, immediate release tablets, bilayer tablets, enteric coated tablets, sustained release tablets.

In another aspect of the invention, one or more anti-diabetic agents is selected from the group consisting of dipeptidyl peptidase IV (DP-IV) inhibitors; insulin sensitizers selected from the group consisting of (i) PPARy agonists, other PPAR ligands, PPAR dual agonists, and PPAR agonists, (ii) biguanides, and (iii) protein tyrosine phosphatase-IB (PTP-1B) inhibitors; insulin or insulin mimetics; sulfonylureas or other insulin secretagogues; alpha-glucosidase inhibitors; glucagon receptor agonists; GLP-1 receptor agonists, and sodium glucose transport protein 2 (SGLT2) inhibitors.

In another aspect of the invention, one or more anti-diabetic agents include, but are not limited to buformin, phenformin, acarbose, ciglitazone, darglitazone, englitazone, pioglitazone, rosiglitazone, troglitazone, acetohexamide, carbutamide, tolbutamide, tolazamide, glibenclamide, gliclazide, gliplizide, miglitol, voglibose, mitiglinide, repaglinide, nateglinide, glimepride, gliclazide, glyclopyramide, chlorpropamide, tolbutamide, phenformin, anagliptin, gemigliptin, alogliptin, sitagliptin, linagliptin, saxagliptin, vildagliptin, teneligliptin, rosiglitazone, pioglitazone, troglitazone, faraglitazar, saroglitazar, englitazone, darglitazone, isaglitazone, zorglitazone, liraglutide, muraglitazar, peliglitazar, tesaglitazar, cangliflozin, dapagliflozin, empagliflozin, remogliflozin, sergliflozin, tofogliflozin.

In other aspects of the invention, reconstitutable, single use antidiabetic composition can be administered orally. The reconstitutable, single use antidiabetic composition is to be filled in the sachet.

In one aspect of the present invention, reconstitutable, single use antidiabetic composition is filled in the sachet in the form of coated pellets, beads, granules, powders, spheroids or tablets and mixture thereof. The process for producing a single use antidiabetic composition comprises palletisation part and granules part.

The term “Sachets” used herein means single-use sachets are disposable packaging materials used to hold small amounts or quantities of products that can be used within a single sitting.

In another aspect of the invention, reconstitutable, single use antidiabetic composition which is present in the sachet administered by reconstitution of pellets, powder, beads, granules, spheroids in the reconstituted media such as water, fresh juices or soft food. Reconstitutable, single use antidiabetic composition is well mixed or stirred in reconstituted media such as water, fresh juices before administration. Furthermore, Reconstitutable, single use antidiabetic composition can be sprinkled on soft food wherein soft food consist of foods that are easily chewed and digested. These foods may be chopped, ground, smashed, pureed, and moist.

Another aspect of the present invention is to provide a single use composition comprising
a. metformin or pharmaceutically acceptable salt as an extended release composition
b. optionally one or more antidiabetic agent(s); and

optionally pharmaceutical acceptable excipients; wherein osmogents does not control the release characteristics of metformin or pharmaceutically acceptable salt.

The term “osmogent” refers to all pharmaceutically acceptable inert water-soluble compounds that can imbibe water and/or aqueous biological fluids.

Osmogents which does not control the release characteristics of metformin or pharmaceutically acceptable salt is xylitol, mannitol, glucose, lactose, sucrose, and sodium chloride.

More precisely, the present invention particularly relates to single use composition comprising
a. metformin or pharmaceutically acceptable salt as an extended release composition as portion 1
b. external phase as portion 2 optionally one or more antidiabetic agent(s):

wherein extended release portion 1 and portion 2 is filled into the single dose sachet at different stages.

In another embodiment of the present invention, portion 1 of single use metformin extended release composition is in the form of pellets, beads, granules, powder, spheroids and mixture thereof. Further portion 1 of single use metformin extended release composition contains free flowing powder which comprises palletisation part and granules part.

In another embodiment of the present invention, portion 2 of the said invention is an immediate release one or more antidiabetic agents and pharmaceutically acceptable excipients.

### Filling of Reconstitutable, Single Use Antidiabetic Compositions:

In one embodiment of the present invention, filling of Reconstitutable, single use antidiabetic compositions at different stages as follows:
a. before sealing of sachet portion 1 of single use metformin extended release composition comprises palletisation part and granules part are filled in sachet.
b. further in addition to step a. portion 2 is filled in a sachet
c. sealing of the sachet

Further aspect of the present invention is to provide a single use pharmaceutical composition comprising
a. metformin or pharmaceutically acceptable salt as an extended release composition
b. optionally one or more antidiabetic agent(s); and

wherein dose uniformity of active ingredients is as per USP.

The term “dose uniformity” as used herein, as per USP Chapter 905 defined as “the degree of uniformity in the amount of the drug substance among dosage units.”

In another aspect of the present invention, single use composition comprising
a. metformin or pharmaceutically acceptable salt as an extended release composition
b. optionally one or more antidiabetic agent(s); and

wherein dosing accuracy for active ingredients per dose is between 95% and 105%.

In another aspect of the invention, single use composition is having accurate dose of extended release composition of metformin or pharmaceutically acceptable salt. Dosing accuracy of single dose composition is independent of 500 mg, 750 mg and 1000 mg of sachet.

In one embodiment of the present invention, single use metformin extended release composition may be in the form of pellets made up of inert spheres. Inert spheres selected from the group consisting of microcrystalline cellulose spheres, sugar spheres, dibasic calcium phosphate spheres, silica spheres, a tartaric acid spheres.

According to an aspect of the present invention, single use composition comprising
a.) metformin or pharmaceutically acceptable salt as an extended release composition contains pellet
b.) optionally one or more antidiabetic agent(s), wherein pellet having size not more than 850 μm

According to another aspect of the present invention, single use composition comprising metformin or pharmaceutically acceptable salt as an extended release composition contains pellet typically has size in the range from 150 μm to 850 μm.

According to a further aspect of the present invention, coating compositions comprises controlled release polymers, binders and plasticizers.

According to the present invention, said controlled release polymers can be selected from the group consisting of cellulosic polymers such as ethyl cellulose, hydroxypropyl methylcellulose; Surelease ARC; hydroxypropylcellulose, cellulose acetate, sodium alginate, carbomer, sodium carboxymethyl cellulose, xanthan gum, guargum, locust bean gum, polyvinyl alcohol, cellulose acetate, cellulose acetate succinates, cellulose acetate phthalates, polyvinyl acetate, polyvinyl succinates, methacrylic acid esters neutral polymer, hydroxypropyl methyl cellulose phthalate, hydroxypropyl methyl cellulose succinates, hydrogenated castor oil, waxes and mixture thereof.

In another embodiment of the present invention, binders are selected from the group consisting of starch, polyvinyl pyrrolidone/povidone, pregelatinized starch, hydroxypropylmethyl cellulose, hydroxyethyl cellulose, methyl cellulose, sodium carboxymethyl cellulose, gums, acrylate polymers, and mixtures thereof.

In another embodiment of the present invention, plasticizers are selected from the group consisting of dibutylsebacate, triethyl citrate, triacetin, acetylated triacetin, tributyl citrate, glyceryl tributyrate, sorbitol, diethyl oxalate, diethyl phthalate, diethyl malate, diethylmalonate, dioctyl phthalate, and combinations thereof.

In another embodiment of the present invention, flavoring agents are selected from the group consisting of pineapple, strawberry, raspberry, mango, passion fruit, kiwi, apple, pear, peach, apricot, cherry, grape, banana, cranberry, blueberry, black currant, red currant, gooseberry, lingon berries, cumin. thyme, basil, camille, parsley, chamomile, tarragon, lavender, dill, bergamot, salvia, aloe vera balsam, spearmint, eucalyptus, and combination thereof.

In one of the embodiment, reconstitutable, single use antidiabetic composition can be filled into sachet and packed in child resistance sachet/CRC pack. Further, single dose sachet gives long term storage stability of single use composition of metformin or pharmaceutically acceptable salt as an extended release composition.

According to an aspect of the present invention, a single use composition comprising
a. metformin or pharmaceutically acceptable salt
b. optionally one or more antidiabetic agent(s); and
optionally pharmaceutical acceptable excipients; wherein the single use composition is in the form of dispersible tablets which is reconstituted just before consumption.

According to further aspect of the present invention, dispersible tablets packed in child resistance sachets/CRP pack.

### EXAMPLES

There are six different reconstitutable, single use antidiabetic compositions were prepared as follows:

### Examples 1 (F-1) to Example 6 (F-6)

| | F1 | F2 | F3 | F4 | F5 | F6 |
|---|---|---|---|---|---|---|
| Ingredients | mg/unit | | | | | |
| Part A: Drug | | | | | | |
| coated pellets | | | | | | |
| Starter Pellets | | | | | | |
| MCC Sphere | 560 | 560 | 560 | 560 | 560 | 560 |
| Drug coating | | | | | | |
| Metformin | 1000 | 900 | 1000 | 900 | 1000 | 1000 |
| Hydrochloride | | | | | | |
| Hydroxy methyl | 50 | 45 | 50 | 45 | 50 | 50 |
| propyl cellulose | | | | | | |
| Povidone | 35 | 31.5 | 35 | 31.5 | 35 | 35 |
| Purified water | Q.S. | Q.S. | Q.S. | Q.S. | Q.S. | Q.S. |
| Extended | | | | | | |
| Release Coating | | | | | | |
| Ethyl cellulose | 500 | 450 | 700 | 700 | 500 | 700 |
| 20 Cps | | | | | | |
| Ethyl cellulose | 500 | 450 | 0 | 0 | 500 | 0 |
| 100 Cps | | | | | | |
| Dibutyl sebacate | 28 | 25.2 | 28 | 28 | 28 | 28 |
| Acetone | Q.S. | Q.S. | Q.S. | Q.S. | Q.S. | Q.S. |
| Purified water | Q.S. | Q.S. | Q.S. | Q.S. | Q.S. | Q.S. |
| Drug coating II | | | | | | |
| Metformin | 0 | 100 | 0 | 100 | 0 | 0 |
| Hydrochloride | | | | | | |
| HPMC | 0 | 5 | 0 | 5 | 0 | 0 |
| Povidone | 0 | 5 | 0 | 5 | 0 | 0 |
| Purified water | 0 | Q.S. | 0 | Q.S. | 0 | 0 |
| Total weight | 2673.0 | 2570.2 | 2373.0 | 2373.0 | 2673.0 | 2373.0 |
| Part B: | | | | | | |
| Dispersing agent | | | | | | |
| Dapagliflozin | 0 | 0 | 0 | 0 | 5 | 5 |
| Lactose | 499 | 499 | 499 | 499 | 494 | 494 |
| Monohydrate | | | | | | |
| Pregelatinized | 100 | 100 | 100 | 100 | 100 | 100 |
| starch | | | | | | |
| Sucralose | 10 | 10 | 10 | 10 | 10 | 10 |
| Colloidal silicon | 5 | 5 | 5 | 5 | 5 | 5 |
| dioxide | | | | | | |
| Flavour | 2 | 2 | 2 | 2 | 2 | 2 |
| Purified water | Q.S. | Q.S. | Q.S. | Q.S. | Q.S. | Q.S. |
| Total weight | 616 | 616 | 616 | 616 | 616 | 616 |

### Procedure for Preparation of Examples 1 (F-1) to Example 6 (F-6)

1. Dispensing: Metformin hydrochloride, MCC [Microcrystalline cellulose] spheres, hydroxypropyl methylcellulose, and povidone were dispensed
2. Preparation of Drug Coating Solution I: Metformin hydrochloride, hydroxypropyl methylcellulose, povidone were dissolved in purified water. Further, MCC spheres were coated with drug coating solution using fluidized bed processor.
3. Preparation of polymer coating solution: Ethyl cellulose 20 cps, Ethyl cellulose 100 cps, dibutyl sebacate were dissolved in acetone and purified water. Drug coated pellets prepared in step 2 were coated with polymer coating solution using fluidized bed processor.
4. Curing of polymer coated pellets: polymer coated pellets as prepared in step 3 were cured.
5. Preparation of Drug Coating Solution II: Metformin hydrochloride, hydroxypropyl methylcellulose, povidone were dissolved in purified water. Polymer coated pellets as prepared in step 3 were coated with drug coating solution II using fluidized bed processor.
6. Drying: Drying of drug coated pellets prepared in step 5 were dried for 30 minutes.
7. Granules preparation: Dispensing of Dapagliflozin, lactose monohydrate, pregelatinized starch, sucralose, colloidal silicon dioxide, and flavouring agent was done. Dapagliflozin, lactose monohydrate, pregelatinized starch, sucralose, colloidal silicon dioxide, and flavouring agent were co-sifted using suitable mesh and mixture were formed. Further purified water was added as granulating fluid to the mixture and granulated using rapid mixer granulator. Granules were dried, sifted and blended.
8. Drug coated pellets as prepared in step 6 and granules prepared in step 7 were filled in sachets individually and packed in child resistance package.

### Comparison of Dissolution Data of Examples 1 (F-1) to Example 6 (F-6) with RIOMET ER

The in-vitro drug release profile of single use composition of metformin or pharmaceutically acceptable salt performed in USP type II apparatus at 100 rpm, in 1000 ml of phosphate buffer with pH 6.8 at 37° C. The dissolution profile results are shown in the Table 1.

TABLE 1
In-vitro drug release profile
Percentage (%) of the In-Vitro Metformin Release
in USP Type II Apparatus
(Media: Phosphate Buffer, pH 6.8, 1000 mL, 100 rpm )

| Time | | | | | | | |
|---|---|---|---|---|---|---|---|
| (hours) | Innovator | T1 | T2 | T3 | T4 | T5 | T6 |
| 0.5 | 9 | 10 | 12 | 8 | 11 | 12 | 13 |
| 1 | 17 | 18 | 20 | 16 | 17 | 18 | 19 |
| 2 | 28 | 29 | 31 | 27 | 28 | 29 | 39 |
| 4 | 49 | 50 | 52 | 48 | 49 | 50 | 51 |
| 6 | 69 | 70 | 72 | 68 | 69 | 70 | 71 |
| 8 | 84 | 85 | 87 | 83 | 84 | 85 | 82 |
| 10 | 92 | 93 | 95 | 91 | 92 | 93 | 90 |
| 14 | 96 | 97 | 97 | 95 | 91 | 98 | 94 |
| 16 | 99 | 100 | 100 | 98 | 97 | 101 | 100 |

TABLE 2
Sieve Analysis Data of Pellets

| | Retains on | | | Drug Coated |
|---|---|---|---|---|
| Retains on | Screen No | Drug Coated | ER | (DC) II |
| Sieve No (#) | (μm) | (DC) Pellets | Pellets | Pellets |
| 20 | 850 | 0.0 | 0.0 | 0.0 |
| 30 | 600 | 0.0 | 1.0 | 0.0 |
| 40 | 425 | 1.0 | 6.0 | 2.0 |
| 60 | 250 | 9.0 | 53.0 | 55.0 |
| 80 | 180 | 79.0 | 100.0 | 99.0 |
| 100 | 150 | 84.0 | 100.0 | 100.0 |
| Base Plate/ | — | 100.0 | 100.0 | 100.0 |
| 100 pass | | | | |

1.-22. (canceled)

23. A powder for suspension, wherein the powder comprises a single population of pellets and a dispersing agent:
the single population of pellets comprises
a sphere, a first coating layer surrounding the sphere, wherein the first coating layer comprises a first quantity of metformin HCl, a first water soluble polymer and a first binding agent,
a second coating layer surrounding the first coating layer and comprising an extended release polymer and a plasticizer,
a third coating layer comprising a second quantity of metformin, a second water soluble polymer and a second binding agent; and
the dispersing agent comprises granules comprising one or more of a flavoring agent, a sweetener agent and one or more pharmaceutical excipients.

24. The powder for suspension of claim 23, wherein the composition is devoid of an osmogent

25. The powder for suspension of claim 23, wherein a weight ratio of the weight of the single population of pellets in the powder for suspension to the weight of the dispersing agent in the powder for suspension is about 2:1 to about 4.5:1.

26. The powder for suspension of claim 25, wherein the weight ratio of the weight of the single population of pellets to the weight of dispersing agent is about 3.85:1.

27. The powder for suspension of claim 23, wherein a weight ratio of the weight of the first quantity of metformin to the weight of the second quantity of metformin is about 9.5:0.5 to 8.5:1.5.

28. The powder for suspension of claim 23, wherein a weight ratio of the weight of the first quantity of metformin to the weight of the second quantity of metformin is about 9:1.

29. The powder for suspension of claim 23, wherein a weight ratio of the weight of the extended release polymer to the weight of the plasticizer is about 25:1.

30. The powder for suspension of claim 23, wherein the dispersing agent is devoid of metformin.

31. The powder for suspension of claim 23, wherein the particle size of the single population pellets is less than 400 microns as measured by sieve analysis.

32. The powder for suspension of claim 23, wherein at least one of the first water soluble polymer and the second water soluble polymer comprises HPMC.

33. The powder for suspension of claim 23, wherein at least one of the first binder and the second binder comprises povidone.

34. The powder for suspension of claim 23, wherein the extended release polymer comprises ethyl cellulose.

35. The powder for suspension of claim 23, wherein the plasticizer comprises dibutyl sebacate.

36. The powder for suspension of claim 23, wherein the composition has a release profile of metformin of:

| Time (hours) | Release (%) |
|---|---|
| 0.5 | 11 |
| 1 | 17 |
| 2 | 28 |
| 4 | 49 |
| 6 | 69 |
| 8 | 84 |
| 10 | 92 |
| 14 | 91 |
| 16 | 97 |

in USP Type II apparatus, phosphate buffer media, pH 6.8, 1,000 mL and 100 rpm.

37. The powder for suspension of claim 23, wherein the powder for suspension is contained within a sachet.

38. A method of administering metformin, the method comprising:
providing the powder for suspension composition of metformin of claim 23 to an individual in need of metformin;
adding the powder for suspension composition of metformin to a liquid to reconstitute the composition; and
orally ingesting the reconstituted composition.

39. The method of claim 38, wherein the liquid is one or more of water, fruit juice or a soft food.

40. The method of claim 38, wherein the powder for suspension composition of metformin is within a sachet.
